(19) United States
(12) Patent Application Publication
(10) Pub. No.: US 2010/0113875 A1
(43) Pub. Date: May 6, 2010

(54) ENDOSCOPE AND MOVEMENT CONTROL SYSTEM OF THE SAME

(75) Inventors: Byung Ju Yi, Gyeonggi-do (KR); Dong Geol Choi, Seoul (KR); Youngsoo Kim, Seoul (KR)

Correspondence Address: FISH & RICHARDSON P.C., P.O. BOX 1022, MINNEAPOLIS, MN, 55440-1022, US

(73) Assignee: IUCF-HYU (Industry-University Cooperation Foundation Hanyang University), Seoul (KR)

(21) Appl. No.: 12/531,721

(22) PCT Filed: Mar. 19, 2008
(86) PCT No.: PCT/KR2008/001537

§ 371 Date: Sep. 17, 2009

(30) Foreign Application Priority Data
Sep. 19, 2007 (KR) 10-2007-0026478

(51) Int. Cl. A61B 1/04; A61B 1/00

(52) U.S. Cl. 600/110; 600/101

(57) ABSTRACT

Disclosed are an endoscope and a system for controlling movement of the same. The system includes an elastic spring having a predetermined length, a camera mounted on an end of the elastic spring, a plurality of link bodies shaped to fit around the elastic spring and disposed to have a predetermined interval therebetween, a plurality of wires connected with the plurality of link bodies for allowing each of the plurality of link bodies to be connected with each other along a longitudinal direction of the elastic spring, a plurality of driving modules connected with an end of each of the plurality of wires for tightening or loosening each of the plurality of wires, and a control module electrically connected with the plurality of driving modules and the camera for driving the plurality of the driving modules and receiving visual information from the camera.

### TECHNICAL FIELD

The present invention relates to an endoscope, and more particular, to an endoscope and a system for controlling movement of the same in which a body of the endoscope having a predetermined length is flexibly moved, and simultaneously a movement direction of the endoscope is changed.

### BACKGROUND ART

In general, an endoscope device may designate medical equipment used for visually checking and diagnosing abnormal portions of internal organs of the human body. As examples of the medical equipment other than the endoscope, one-dimensional imaging devices such as an X-ray see through device, two-dimensional imaging photographing devices such as an ultrasound scanner, and three-dimensional imaging devices such as an Nuclear Magnetic Resonance-Computerized Tomography (NMR-CT) may be given.

However, the above medical equipment may indirectly measure the inside of the human body from the outside of the human body in such a manner that specific signals created from the inside of the human body are transmitted to the outside, the transmitted signals are associated together or image-processed to thereby acquire information about the inside of the human body. In this regard, the indirectly acquired information as described above may be relatively accurately checked, however, a clear image may be difficult to be acquired due to errors occurring in the received signals and processed signals, a distance with an object to be photographed, that is, the human body, and the like.

Accordingly, there has been developed an endoscope in which a photographing device may be directly inserted to the abnormal portions of the internal organ of the human body to thereby acquire more accurate images, in comparison with a device for image-processing signals transmitted to the outside of the human body to thereby indirectly check the abnormal portions.

A conventional endoscope may include a hose having a predetermined length, a camera mounted at an end of the hose, and a display module for receiving images acquired from the camera and displaying the received image. When the conventional endoscope is inserted passing through an anus or mouth of a patient and advanced to the inside of the internal organ, the conventional endoscope has a problem in that the end of the hose with the camera mounted may be difficult to be bent in a desired direction.

Also, in the conventional art, when the hose of the endoscope is bent towards a desired direction in the internal organ, the hose and the camera may strike against a wall of the internal organ and accordingly inflict damage upon the wall.

### DISCLOSURE OF INVENTION

### Technical Goals

An aspect of the present invention provides an endoscope and a system for controlling movement of the same in which a desired portion in an internal organ of a human body may be accurately observed using the endoscope inserted passing through a mouth or anus of the human body.

An aspect of the present invention provides an endoscope and a system for controlling movement of the same in which a body of the endoscope having a predetermined length may be flexibly bent towards a desired portion in the internal organ of the human body.

### Technical Solutions

According to an aspect of the present invention, there is provided an endoscope, which includes: an elongated and bendable body; a plurality of wires disposed around the body along a longitudinal direction of the body; and a plurality of driving modules connected with an end of each of the plurality of wires for tightening or loosening each of the plurality of wires.

In this instance, the body may be formed to correspond to an entire length of the endoscope, or a portion of the endoscope in which a camera is mounted. Also, the body may be made of a flexible material having a predetermine length. As an example, the body may be formed using an elastic spring having a predetermined length.

Also, a plurality of link structures may be mounted on the body in such a manner as to have a predetermined interval therebetween. In this instance, each of the plurality of link structures may include a link body with a hollow formed therein for receiving the body and including a plurality of inserting holes passing through the link body along a longitudinal direction of the link body for allowing each of the plurality of wires to be inserted therein. In this instance, the link structure may be made of aluminum.

Also, the plurality of inserting holes may be formed on the body to have a predetermined interval therebetween and correspond to the plurality of wires.

In this instance, the plurality of inserting holes may be radially arranged with respect to a center of the link body in such a manner as to have a predetermined interval therebetween.

Also, each of the plurality of driving modules may include a roller connected with an end of each of the plurality of wires being wound around the roller, and a motor for rotating the roller with respect to a center of the roller in forward and backward directions.

Also, a camera for recognizing external visual information may be mounted on an end of the body opposite to another end of the body where the plurality of driving modules is mounted.

According to an aspect of the present invention, there is provided a system for controlling operations of an endoscope, which includes: an elastic spring having a predetermined length; a camera mounted on an end of the elastic spring; a plurality of link bodies shaped to fit around the elastic spring and disposed to have a predetermined interval therebetween; a plurality of wires connected with the plurality of link bodies for allowing each of the plurality of link bodies to be connected with each other along a longitudinal direction of the elastic spring; a plurality of driving modules connected with an end of each of the plurality of wires for tightening or loosening each of the plurality of wires; and a control module electrically connected with the plurality of driving modules and the camera for driving the plurality of the driving modules and receiving visual information from the camera.

In this instance, each of the plurality of the link bodies may include a plurality of inserting holes passing through the link body along a longitudinal direction of the link body for allowing each of the plurality of wires to be inserted therein. Also, each of the plurality of link bodies is made of aluminum.

Also, the plurality of inserting holes may be radially arranged with respect to a center of the link body in such a manner as to have a predetermined interval therebetween.

Also, each of the plurality of driving modules may include a roller connected with an end of each of the plurality of wires being wound around the roller, and a motor for rotating the roller with respect to a center of the roller in forward and backward directions.

Also, in the control module, operation information of the motor of the plurality of driving modules may be predetermined according to information about a bent direction of the elastic spring, and preferably, the control module may drive the motor of the plurality of driving modules according to the predetermined operation information.

### BRIEF DESCRIPTION OF DRAWINGS

FIG. 1 is a cross-sectional diagram illustrating an endoscope and system for controlling movement of the endoscope according to an exemplary embodiment of the present invention; and

FIG. 2 is a perspective diagram illustrating a coupled relation between a body, link structure, and wire according to an exemplary embodiment of the present invention.

### BEST MODE FOR CARRYING OUT THE INVENTION

Reference will now be made in detail to embodiments of the present invention, examples of which are illustrated in the accompanying drawings, wherein like reference numerals refer to the like elements throughout. The embodiments are described below in order to explain the present invention by referring to the figures.

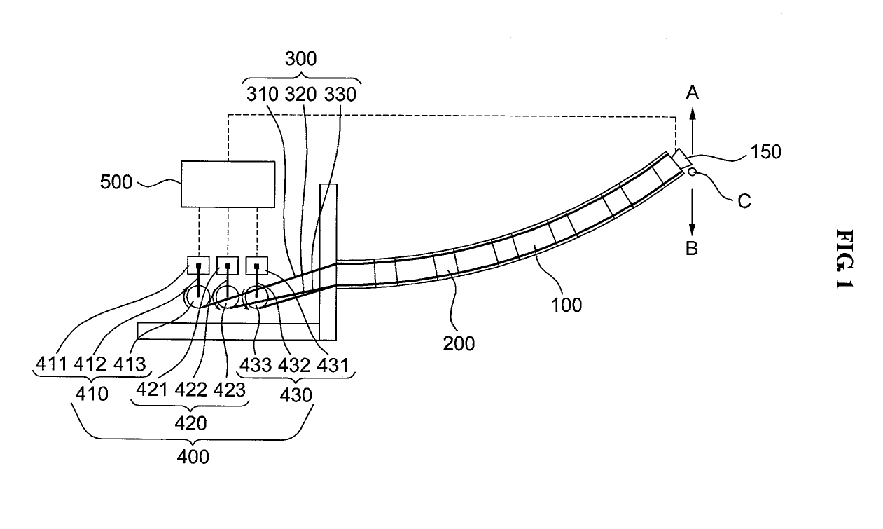

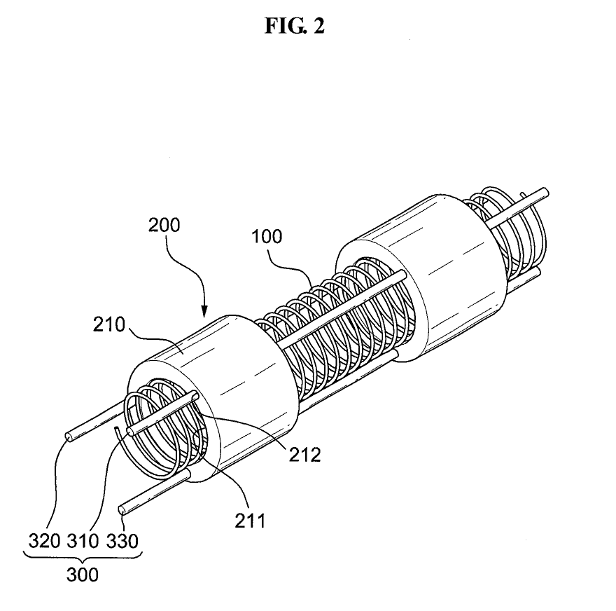

FIG. 1 is a cross-sectional diagram illustrating an endoscope and system for controlling movement of the endoscope according to an exemplary embodiment of the present invention, and FIG. 2 is a perspective diagram illustrating a coupled relation between a body, link structure, and wire according to an exemplary embodiment of the present invention.

Referring to FIG. 1, the endoscope of the present exemplary embodiment of the invention may include an elongated and bendable body 100, and a plurality of link structures 200 shaped to fit around the body 100 and disposed to have a predetermined interval therebetween.

The body 100 may be elongated to have a predetermined length and provided with various structures and materials enabled to be elongated and bent. For example, according to the present exemplary embodiment of the invention, the body 100 is provided with a spring elastic body, and the spring elastic body may be vertically and horizontally bent.

The body of the spring elastic body may have a restoring force to return to an original position against the twisting by itself Accordingly, when intending to return the original position of the body in a direction, or to bend the body in the opposite direction, the restoring force of the spring elastic body may support the restoration and bend of the body. Also, an operator or a user may readily manipulate the endoscope, and permit a driving module required for the restoration to be small and simple.

Also, the body 100 of the spring elastic body may be freely bendable by itself, and a number of wires enabled to be embedded in the body may be increased without being restricted by three or four wires, thereby enabling elaborate manipulation of the body.

Each of the plurality of the link structures 200 may include a link body 210 with a hollow 211 formed therein for receiving the body 100, and including a plurality of inserting holes 212 passing through the link body 210 for allowing each of the plurality of wires 300 to be inserted therein. In this instance, each of the plurality of inserting holes 212 may be formed along a longitudinal direction of the link body 210 and have a diameter allowing each of the plurality of wires to pass through each of the plurality of inserting holes 212. Here, each of the plurality of link structures 200 including the link body 210 having the plurality of inserting holes 212 is provided along a longitudinal direction of the body 100 in such a manner as to have an even or uneven interval therebetween. Each of the plurality of link structures 200 may be made of aluminum or a metal including aluminum, and also made of nonmetal material as long as physical characteristics are similar with the plurality of link structures 200.

Each of the plurality of wires 300 is inserted into each of the plurality of inserting holes 212 of the link body 210 along a longitudinal direction of the body 100.

As illustrated in FIG. 1, an end of each of the plurality of wires 300 is connected with each of a plurality of driving modules 400. The plurality of driving modules 400 may function to tighten or loosen each of the plurality of wires 300. As an example, the plurality of driving modules 400 may include a first driving module 410, a second driving module 420, and a third driving module 430.

The first to third driving modules 410, 420, and 430 may be driven by receiving a power from the outside. In this instance, each of the driving modules may include forward and backward motors for controlling tension. The first to third driving modules 410, 420, and 430 may include: first, second, and third motors 411, 421, and 431 rotatable in forward and backward directions; first, second, and third rotation shafts 412, 422, 432 each connected with the first, second, and third motors and rotatable in forward and backward directions; and first, second, and third rollers 413, 423, and 433 each connected with the first, second, and third rotating shafts 412, 422, and 432 and rotatable in forward and backward directions.

The first, second, and third rollers 413, 423, and 433 may be connected with an end of the first, second, and third wires 310, 320, and 330, respectively. Accordingly, the first, second, and third wires 310, 320, and 330 may be tightened or loosened by the rotation of the first, second, and third rollers 413, 423, and 433.

Here, the plurality of wires 300 may be preferably provided by three wires as described above. Also, according to the present exemplary embodiment of the invention, tension and relaxation of the plurality of wires may be adjusted using the rollers. However, a separate actuator such as a cylinder may be used for adjusting the tension and relaxation of the plurality of wires, as necessary. Alternatively, the plurality of wires itself may be made of a flexible material.

Referring again to FIG. 1, the plurality of driving modules 400 may include the first driving module 410 connected with an end of the first wire 310, the second driving module 420 connected with an end of the second wire 320, and the third driving module 430 connected with an end of the third wire 330. Also, a camera 150 for acquiring external visual information is mounted on an end of the body 100.

The above described endoscope may include a system for controlling movement of the endoscope.

The system for controlling movement of the endoscope according to the present invention may include a control module 500 electrically connected with the first, second, and third driving modules 410, 420, and 430.

An operator or user of the system for controlling movement of the endoscope may control movement of the endoscope using manufacturing devices such as a joystick, buttons, and the like, and thus controlling information about a bent direction of the body 100. In the control module 500, operation information of the motors 411, 421, and 431 of the plurality of driving modules 400 may be predetermined according to the information about the bent direction of the body 100.

Here, the information about the bent direction of the body 100 may be direction information about a direction in which the body 100 is bent, as illustrated in FIG. 1, and for example, may be information indicating that the body 100 is bent in an arrow direction A.

Accordingly, the control module 500 may drive the motors 411, 421, and 431 of the driving modules 400 according to the predetermined operation information.

Specifically, as illustrated in FIG. 1, when the body 100 is bent in the arrow direction A, the control module 500 may partially or completely drive the first, second, and third driving modules 410, 420, and 430.

As an example, for the purpose of following the bend of the body 100 in the arrow direction A, the first driving module 410 may drive the first motor 411 to thereby tighten the first wire 310 wound on the first roller 413. Also, the second and third driving modules 420 and 430 may drive the second and third motors 421 and 431, respectively, to thereby loosen the second and third wires 320 and 330 each wound on the second and third rollers 423 and 433.

Specifically, when the body 100 of the endoscope is bent in the arrow direction A illustrated in FIG. 1, the control module 500 may drive all of the first, second, and third driving modules 410, 420, and 430. The first driving module 410 may drive the first motor 411 to thereby tighten the first wire 310 wound on the first roller 413.

Also, the second and third driving modules 420 and 430 may drive the second and third motors 421 and 431, respectively, to thereby loosen the second and third wires 320 and 330 wound on the second and third rollers 423 and 433.

In this instance, the elastic spring, that is, the body 100 may be contracted towards the first wire 310.

Also, when the body 100 is bent in an arrow direction B as illustrated in FIG. 1, the control module 500 may drive all of the first, second, and third driving modules 410, 420, and 430.

The third driving module 430 may drive the third motor 431 to thereby tighten the third wire 330 wound on the third roller 433. Also, the first and second driving modules 410 and 420 may drive the first and second motors 411 and 421 to thereby loosen the first and second wires 310 and 320 wound on the first and second rollers 413 and 423.

In this instance, the elastic spring, that is, the body 100 may be contracted towards the third wire 330.

Also, when the body 100 is bent in an arrow direction C (perpendicular to the arrow direction A) as illustrated in FIG. 1, the control module 500 may drive all of the first, second, third driving modules 410, 420, and 430.

The second driving module 420 may drive the second motor 421 to thereby tighten the second wire 320 wound on the second roller 423. Also, the first and third driving modules 410 and 430 may drive the first and third motors 411 and 431 to thereby loosen the first and third wires 310 and 330 wound on the first and third rollers 413 and 433.

In this instance, the elastic spring, that is, the body 100 may be contracted towards the second wire 320.

As described above, the body 100 of the endoscope according to the present exemplary embodiment of the invention may be bent in a bendable direction as desired by tightening the wires 300 disposed in the bent direction of the driving modules 400 using the driving modules 400 operated by driving signals of the control module 500, and the plurality of wires 300 connected with the driving modules 400 and contracted and relaxed.

Referring to FIG. 1, when the body 100 is bent towards between the arrow directions A and B, the control module 500 may drive the first and second driving modules 410 and 420 to thereby simultaneously tighten the first and second wires, or may drive the third driving module 430 to thereby loosen the third wire 330.

Also, when the body 100 is bent towards between the arrow directions B and C, the control module 500 may drive the second and third driving modules 420 and 430 to thereby simultaneously tighten the second and third wires 320 and 330, or may drive the first driving module 410 to thereby loosen the first wire 310.

As described above, according to the present invention, a desired portion to be observed in the internal organ of the human body may be accurately observed using the endoscope inserted passing through a mouth or anus of the human body. In this instance, the endoscope is operated such that the plurality of driving modules is used in the outside of the human body to thereby tighten or loosen the wires connected with the plurality of driving modules.

According to the present invention, the body of the endoscope having a predetermined length is flexibly bent towards a desired portion in the internal organ of the human body using the elastic spring, thereby preventing damage of the internal organ such as when striking against the wall of the internal organ.

Although a few embodiments of the present invention have been shown and described, the present invention is not limited to the described embodiments. Instead, it would be appreciated by those skilled in the art that changes may be made to these embodiments without departing from the principles and spirit of the invention, the scope of which is defined by the claims and their equivalents.

1. An endoscope, comprising:
an elongated and bendable body;
a plurality of wires disposed around the body along a longitudinal direction of the body; and
a plurality of driving modules connected with an end of each of the plurality of wires for tightening or loosening each of the plurality of wires.

2. The endoscope of claim 1, wherein the body is an elastic spring having a predetermined length.

3. The endoscope of claim 1, wherein a plurality of link structures is mounted on the body in such a manner as to have a predetermined interval therebetween, and each of the plurality of link structures includes a link body with a hollow formed therein for receiving the body and including a plurality of inserting holes passing through the link body along a longitudinal direction of the link body for allowing each of the plurality of wires to be inserted therein.

4. The endoscope of claim 3, wherein the link structure is made of aluminum.

5. The endoscope of claim 3, wherein the plurality of inserting holes is radially arranged with respect to a center of the link body in such a manner as to have a predetermined interval therebetween.

6. The endoscope of claim 1, wherein each of the plurality of driving modules includes a roller connected with an end of each of the plurality of wires being wound around the roller, and a motor for rotating the roller with respect to a center of the roller in forward and backward directions.

7. The endoscope of claim 1, wherein a camera for recognizing external visual information is mounted on an end of the body opposite to another end of the body where the plurality of driving modules is mounted.

8. An endoscope, comprising:
a body being an elastic spring having a predetermined length;
a plurality of wires disposed around the body along a longitudinal direction of the body; and
a plurality of driving modules connected with an end of each of the plurality of wires for tightening or loosening each of the plurality of wires,
wherein a plurality of link structures made of aluminum is mounted on the body in such a manner as to have a predetermined interval therebetween, each of the plurality of link structures includes a link body with a hollow formed therein for receiving the body and including a plurality of inserting holes passing through the link body along a longitudinal direction of the link body for allowing each of the plurality of wires to be inserted therein, the plurality of inserting holes being radially arranged with respect to a center of the link body in such a manner as to have a predetermined interval therebetween, each of the plurality of driving modules includes a roller connected with an end of each of the plurality of wires being wound around the roller and a motor for rotating the roller with respect to a center of the roller in forward and backward directions, and a camera for recognizing external visual information is mounted on an end of the body opposite to another end thereof where the driving modules are mounted.

9. A system for controlling operations of an endoscope, the system comprising:
an elastic spring having a predetermined length;
a camera mounted on an end of the elastic spring;
a plurality of link bodies shaped to fit around the elastic spring and disposed to have a predetermined interval therebetween;
a plurality of wires connected with the plurality of link bodies for allowing each of the plurality of link bodies to be connected with each other along a longitudinal direction of the elastic spring;
a plurality of driving modules connected with an end of each of the plurality of wires for tightening or loosening each of the plurality of wires; and
a control module electrically connected with the plurality of driving modules and the camera for driving the plurality of the driving modules and receiving visual information from the camera.

10. The system of claim 9, wherein each of the plurality of the link bodies includes a plurality of inserting holes passing through the link body along a longitudinal direction of the link body for allowing each of the plurality of wires to be inserted therein.

11. The system of claim 10, wherein each of the plurality of link bodies is made of aluminum.

12. The system of claim 11, wherein the plurality of inserting holes is radially arranged with respect to a center of the link body in such a manner as to have a predetermined interval therebetween.

13. The system of claim 9, wherein each of the plurality of driving modules includes a roller connected with an end of each of the plurality of wires being wound around the roller, and a motor for rotating the roller with respect to a center of the roller in forward and backward directions.

14. The system of claim 13, wherein, in the control module, operation information of the motor of the plurality of driving modules is predetermined according to information about a bent direction of the elastic spring, and the control module drives the motor of the plurality of driving modules according to the predetermined operation information.

15. A system for controlling operations of an endoscope, the system comprising:
an elastic spring having a predetermined length;
a camera mounted on an end of the elastic spring;
a plurality of link bodies shaped to fit around the elastic spring and disposed to have a predetermined interval therebetween;
a plurality of wires connected with the plurality of link bodies for allowing each of the plurality of link bodies to be connected with each other along a longitudinal direction of the elastic spring;
a plurality of driving modules connected with an end of each of the plurality of wires for tightening or loosening each of the plurality of wires; and
a control module electrically connected with the plurality of driving modules and the camera for driving the plurality of the driving modules and receiving visual information from the camera,
wherein each of the plurality of the link bodies includes a plurality of inserting holes passing through the link body along a longitudinal direction of the link body for allowing each of the plurality of wires to be inserted therein, the plurality of inserting holes is radially arranged with respect to a center of the link body in such a manner as to have a predetermined interval therebetween, each of the plurality of driving modules includes a roller connected with an end of each of the plurality of wires being wound around the roller, and a motor for rotating the roller with respect to a center of the roller in forward and backward directions, operation information of the motor of the plurality of driving modules is predetermined, in the control module, according to information about a bent direction of the elastic spring, and the control module drives the motor of the plurality of driving modules according to the predetermined operation information.
